(19) United States
(12) Patent Application Publication
(10) Pub. No.: US 2026/0191555 A1
(43) Pub. Date: Jul. 9, 2026

(54) CATHETER WITH EXPANDABLE SCORING MEMBERS

(71) Applicant: Boston Scientific Scimed, Inc., Maple Grove, MN (US)

(72) Inventors: Brady Scott Logan, Monticello, MN (US); David L Friesen, Otsego, MN (US); Ryan Hendrickson, Albertville, MN (US); Derek Kenneth Larson, Golden Valley, MN (US); Gary John Pederson, JR., Hanover, MN (US)

(73) Assignee: Boston Scientific Scimed, Inc., Maple Grove, MN (US)

(21) Appl. No.: 19/443,170

(22) Filed: Jan. 8, 2026

Related U.S. Application Data
(60) Provisional application No. 63/743,290, filed on Jan. 9, 2025.

(51) Int. Cl. A61B 17/3207; A61B 17/00

(52) U.S. Cl. CPC A61B 17/320725; A61B 2017/00867

(57) ABSTRACT

An example catheter comprises a catheter shaft and a sheathed scoring element, the sheathed scoring element including: shape memory elements that are configured to move between an constrained condition and a radially expanded condition; scoring members located on a portion of the shape memory elements; and a deployment sheath that is configured to move relative to the scoring members and the shape memory elements between: a delivery condition where the scoring members and at least a portion of the shape memory elements are located in the deployment sheath; and a scoring condition where: at least the portion of the shape memory elements is located distal to a distal end of the deployment sheath and the shape memory elements are configured in the expanded condition; and the scoring members are located distal to a distal end of the deployment sheath and are configured to contact a target site.

### CROSS-REFERENCE TO RELATED APPLICATION

This application claims the benefit of U.S. Provisional Patent Application Serial No. 63/743,290, filed January 9, 2025, entitled " CATHETER WITH EXPANDABLE SCORING MEMBERS”, which is incorporated by reference herein in its entirety.

### TECHNICAL FIELD

The disclosure pertains to medical catheters with expandable scoring members. More particularly, the disclosure pertains to catheter systems and apparatuses including sheathed self-expanding shape memory elements with scoring members thereon.

### BACKGROUND

Arterial blockages, which are also called stenosis, lesions, stenotic lesions, etc., are typically caused by the build-up of atherosclerotic plaque on the inside wall of an artery. In fact, several such stenoses may occur contiguously within a single artery. This can result in a partial, or even complete, blockage of the artery. As a result of the danger associated with a blockage, several methods and procedures have been developed to treat stenoses. One such method is an angioplasty procedure which uses an inflatable balloon to dilate the blocked artery. Such approaches typically radially expand the inflatable balloon at a substantially fixed or static location in an effort to disrupt (e.g., crack) a lesion.

Angioplasty balloons have enjoyed widespread acceptance in the treatment of stenoses. The efficacy of the dilation of a stenosis may be enhanced by first, or simultaneously, incising the material that is creating the stenosis. Consequently, developments have been made to equip angioplasty balloons with cutting edges, or atherotomes, which are intended to incise a stenosis during the dilation procedure. For example, inflatable angioplasty medical cutting balloons having a number of atherotomes mounted longitudinally on the surface of the balloon may be employed. Upon inflation of the medical cutting balloon, the atherotomes induce a series of longitudinal cuts into the surface of the stenotic material as the balloon expands to dilate the stenosis. As a result of such cuts, the stenosis is more easily dilated, and the likelihood of damaging the artery during dilation is reduced. If a stent is required, the risk of stent under expansion is reduced if these balloons are used for lesion preparation, as arterial plaque is modified and calcified lesions are disrupted.

In some instances, it may be desirable to supplement a cutting balloon catheter with another scoring device, or use a scoring device instead of a cutting balloon catheter, to enhance vessel compliance, facilitate drug uptake, crack calcified plaque, etc. Accordingly, there is an ongoing need for improved cutting or scoring apparatus and systems such as those that are suitable for atherectomy or other medical procedures.

### SUMMARY

In some aspects, the present disclosure pertains to a catheter for treatment of a vessel lesion. The catheter includesa catheter shaft anda sheathed scoring element. The sheathed scoring element includes shape memory elements that are configured to move between a constrained condition and a radially expanded condition. Scoring members are located on a portion of the shape memory elements. A deployment sheath is provided that is configured to move relative to the scoring members and the shape memory elements between a delivery condition where the scoring members and at least a portion of the shape memory elements are located in the deployment sheath, and a scoring condition where at least the portion of the shape memory elements is located distal to a distal end of the deployment sheath and the shape memory elements are configured in the expanded condition; and the scoring members are located distal to a distal end of the deployment sheath and are configured to contact a target site.

In some aspects, which may be used alternatively or in addition to other aspects herein, at least the proximal ends of the shape memory elements are coupled to the catheter shaft.

In some aspects, which may be used alternatively or in addition to other aspects herein, the shape memory elements further comprise cantilevered shape memory elements.

In some aspects, which may be used alternatively or in addition to other aspects herein, the shape memory elements are formed of shape memory material that is heat set to the expanded condition.

In some aspects, which may be used alternatively or in addition to other aspects herein, the shape memory elements have a pointed distal tip configured to dissect tissue at the target site.

In some aspects, which may be used alternatively or in addition to other aspects herein, the shape memory material further comprises nitinol that is heat-set to the expanded condition.

In some aspects, which may be used alternatively or in addition to other aspects herein, the shape memory elements have the same size and same shape; and the scoring members have the same size and same shape.

In some aspects, which may be used alternatively or in addition to other aspects herein, the scoring members are offset proximally a distance from a distal end of the shape memory elements, are offset distally a distance from a proximal end of the shape memory elements, or both.

In some aspects, which may be used alternatively or in addition to other aspects herein, the scoring members further comprise a scoring wire, a scoring blade, or combinations thereof.

In some aspects, which may be used alternatively or in addition to other aspects herein, the scoring members are disposed on an outer surface of the shape memory elements, and wherein the outer surface is substantially planar.

In some aspects, which may be used alternatively or in addition to other aspects herein, the scoring members extend a distance radially from the outer surface of the shape memory elements.

In some aspects, which may be used alternatively or in addition to other aspects herein, the catheter includes an expansion member configured to impart a radial force on an innermost surface of the portion of the shape memory elements when the deployment sheath is in the scoring condition and the shape memory elements are in the expanded condition.

In some aspects, which may be used alternatively or in addition to other aspects herein, the expansion member is a basket, balloon, a coil or a scaffold.

In some aspects, which may be used alternatively or in addition to other aspects herein, the expansion member is a basket.

In some aspects, which may be used alternatively or in addition to other aspects herein, the basket is formed of a shape memory material.

In some aspects, which may be used alternatively or in addition to other aspects herein, the expansion member is a balloon.

In some aspects, which may be used alternatively or in addition to other aspects herein, the expansion member is a coil.

In some aspects, which may be used alternatively or in addition to other aspects herein, an expansion member is coupled to an inner shaft, wherein the inner shaft is disposed within and is configured to translate longitudinally relative to the catheter shaft. The expansion member may be configured to impart a radial force on an innermost surface of the portion of the shape memory elements when the deployment sheath is in the scoring condition and the shape memory elements are in the expanded condition.

In some aspects, which may be used alternatively or in addition to other aspects herein, the expansion member is configured to expand and impart the radial force on the innermost surface of the portion of the shape memory elements responsive to longitudinal translation of the inner shaft relative to the catheter shaft.

In some aspects, which may be used alternatively or in addition to other aspects herein, a first end of the expansion member is coupled to the inner shaft and wherein a second opposing end of the expansion member floats on the inner shaft.

In some aspects, which may be used alternatively or in addition to other aspects herein, a portion of the expansion member is positioned distal to a distal tip of the shape memory elements when the expansion member is in an unexpanded condition, and the portion of the expansion member is positioned proximal to the distal tip and in contact with the innermost surface of the portion of the shape memory elements responsive to the longitudinal translation of the inner shaft relative to the catheter shaft responsive to the longitudinal translation of the inner shaft relative to the catheter shaft.

In another aspect, a catheter for treatment of a vessel lesion is provided. The catheter includes a catheter shaft and a sheathed scoring element coupled to the catheter shaft. The sheathed scoring element includes shape memory elements formed of a shape memory material that is configured to move between a constrained condition and a radially self-expanded condition. Scoring members are located on a portion of the shape memory elements. A deployment sheath is provided that is configured to move relative to the scoring members and the shape memory elements between a delivery condition where the scoring members and at least a portion of the shape memory elements are located inside of the deployment sheath, and a scoring condition where at least the portion of the shape memory elements is located distal to a distal end of the deployment sheath and the shape memory elements are configured in the radially self-expanded condition and the scoring members are located distal to a distal end of the deployment sheath and are configured to contact a target site. An expansion member is configured to impart a radial force on the portion of the shape memory elements when the deployment sheath is in the scoring condition and the shape memory elements are in the radially self-expanded condition.

In some aspects, which may be used alternatively or in addition to other aspects herein, the shape memory elements are cantilevered substantially longitudinally extending portions of an elongated tubular member, wherein the cantilevered substantially longitudinally extending portions are offset proximally a distance from a distal end region of the elongated tubular member.

In some aspects, which may be used alternatively or in addition to other aspects herein, the scoring members further comprise blades, and wherein the blades are spaced proximally a distance from a distal end of the shape memory elements.

In another aspect, a catheter for treatment of a vessel lesion is provided. The catheter includes a catheter shaft and a sheathed scoring element coupled to the catheter shaft. The sheathed scoring element includes shape memory elements that are formed of a shape memory material and are configured to move between a constrained condition and a radially self-expanded condition. The shape memory elements have a substantially planar outer surface with scoring blades located on a portion of the shape memory elements. The scoring blades are offset at least proximally from distal tips of the shape memory elements and the scoring blades protrude a distance from the substantially planar outer surface of the shape memory elements. A deployment sheath is provided that is axially movable relative to the scoring blades and the shape memory elements between: a delivery condition where the scoring blades and at least a portion of the shape memory elements are located inside of the deployment sheath; and a scoring condition where: at least the portion of the shape memory elements is located distal to a distal end of the deployment sheath and the shape memory elements are configured in the radially self-expanded condition; and the scoring blades are located distal to a distal end of the deployment sheath and are configured to contact a target site; and an expansion member configured to impart a radial force on the portion of the shape memory elements when the deployment sheath is in the scoring condition and the shape memory elements are in the radially self-expanded condition.

In some aspects, which may be used alternatively or in addition to other aspects herein, each scoring blade of the scoring blades is configured to protrude the same distance from the substantially planar outer surface of the shape memory elements, and wherein each of the shape memory elements is formed of a shape memory material that is heat set to the radially self-expanded condition.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

FIG. 1A is a simplified partial longitudinal view of an example catheter including a sheathed scoring element with a deployment sheath in a delivery condition and shape memory elements in a constrained condition disposed in a blood vessel;

FIG. 1B is a simplified partial longitudinal view of the example catheter of FIG. 1A with the shape memory elements in a scoring condition;

FIG. 2A is a simplified partial longitudinal view of the example catheter of FIG. 1B with an example expansion member (basket) in a constrained condition;

FIG. 2B is a simplified partial longitudinal view of the example catheter of FIG. 1B with the example expansion member (basket) in an expanded condition;

FIG. 3A is a simplified partial longitudinal view of the example catheter of FIG. 1B with another example expansion member (balloon) in a constrained condition;

FIG. 3B is a simplified partial longitudinal of the example catheter of FIG. 3A with the expansion member in an expanded condition;

FIG. 4 is a simplified partial longitudinal view of the example catheter of FIG. 1B with another example expansion member (coil) in an expanded condition;

FIG. 5 is a simplified partial longitudinal view of an example catheter including reinforced shape memory elements in an expanded condition;

FIG. 6 is a simplified partial longitudinal view of an example catheter including curved shape memory elements in an expanded condition;

FIG. 7 is a simplified partial longitudinal view of an example catheter including non-linear shape memory elements in an expanded condition;

FIG. 8A is a simplified partial longitudinal view of an example catheter including shape memory elements formed in a portion of an elongated tubular member with the shape memory elements in a constrained condition;

FIG. 8B is a simplified partial longitudinal view of the example catheter of FIG. 8A with the shape memory elements in an expanded condition; and

FIG. 9 is an example of a method employing the example catheters including shape memory elements.

### DETAILED DESCRIPTION

The following description should be read with reference to the drawings wherein like reference numerals indicate like elements throughout the several views. The detailed description and drawings illustrate example embodiments of the claimed invention.

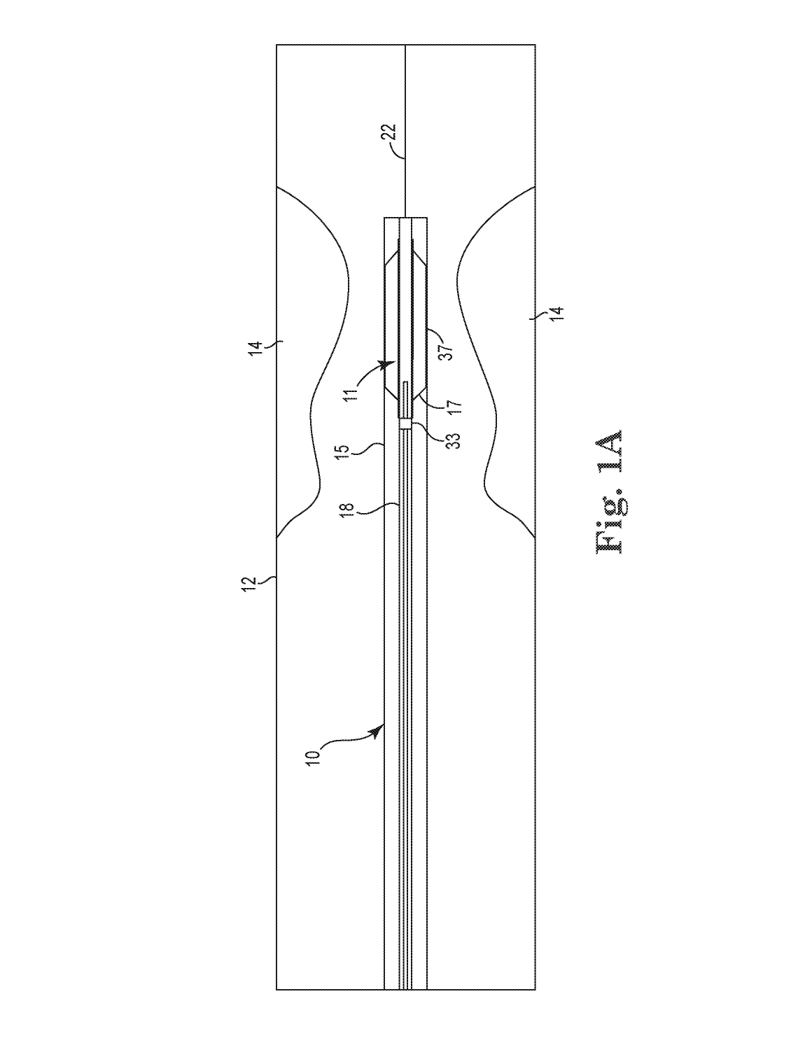

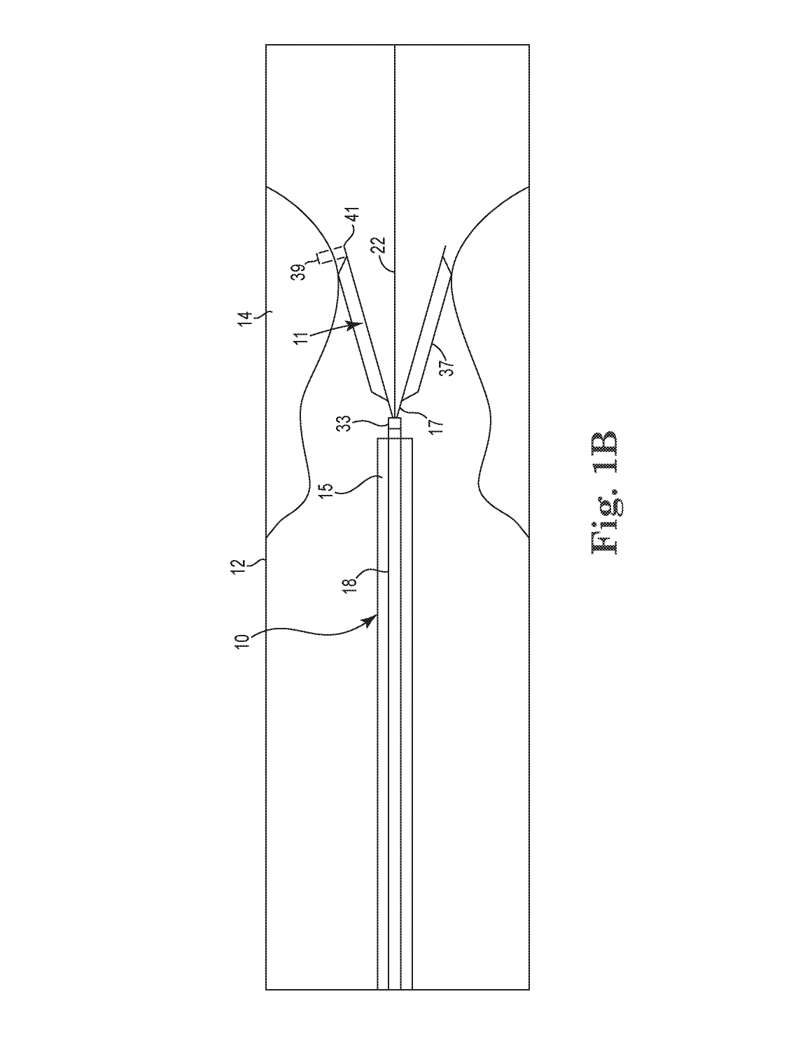

FIG. 1A illustrates a simplified view of an example atherectomy catheter 10 including a sheathed scoring element 11 with shape memory elements 17 in a constrained condition positioned in a blood vessel 12 adjacent to a target site 14, namely an intravascular lesion or stenosis. FIG. 1B is a simplified partial longitudinal view of the example catheter 10 of FIG. 1A with the shape memory elements 17 in a scoring condition (e.g., a radially self-expanded scoring condition).

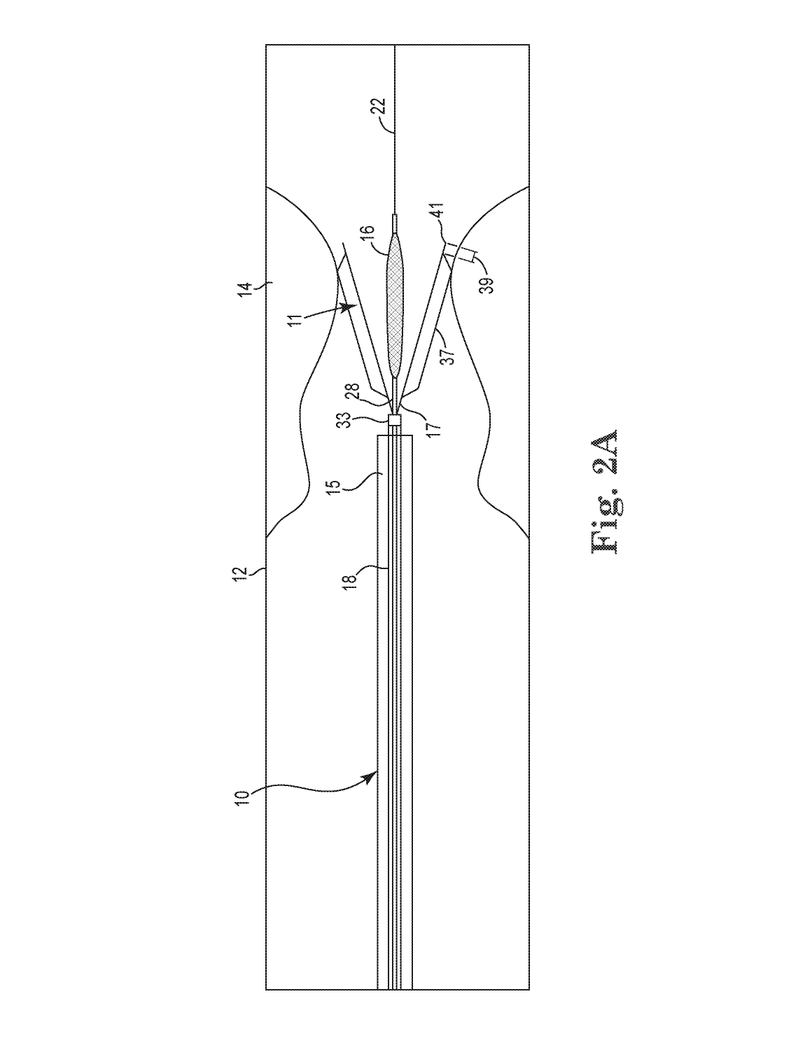

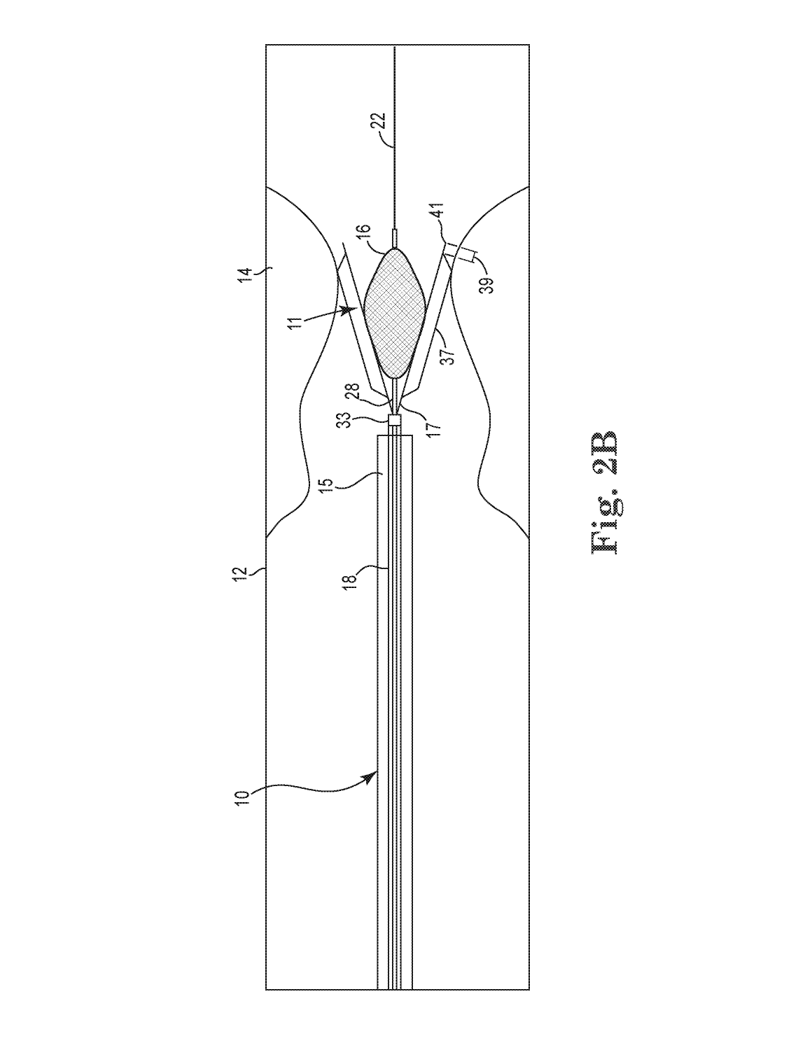

In some embodiments, the sheathed scoring element 11 can be utilized in conjunction with an expansion member. Hence, the systems and methods herein can include the sheathed scoring element and an expansion member. In such instances, the sheathed scoring element is separate from the expansion member 16 (FIGS. 2A-2B, 3A-3B, and 4), unlike some other approaches such as those that employ a cutting balloon with a scoring member affixed to a surface (e.g., an exterior surface) of the balloon in a permanent manner. As used herein, having the components (e.g., the sheathed scoring element 11 and the expansion member 16) be “separate” refers to the components not being coupled together (e.g., permanently coupled together) at least prior to and during delivery to a target site in a vessel. Having the sheathed scoring element 11 be separate from the expansion member 16 can promote aspects herein. For instance, the systems and apparatuses herein can provide improved deliverability to, positioning at, and/or retention at a target site 14 in a blood vessel due at least in part to the sheathed scoring element 11 and the expansion member 16 being separate components (e.g., rather than as an all-in-one device in which the balloon includes a scoring member permanently coupled thereto). That is, in various embodiments the expansion member 16 herein does not include a scoring member (e.g., does not include a scoring member permanently affixed to an exterior surface of the expansion member 16).

Additionally, the system and apparatuses herein at least due in part to the sheathed scoring element 11 and the expansion member 16 being separate components can include shape memory elements 17, can include a larger quantity of scoring members (e.g., five or more scoring members, etc.), and/or can include scoring members that exhibit an improved scoring or cutting force at a target site 14 in a vessel. For instance, due to the use of the sheathed scoring element 11 that include the shape memory elements 17, the catheters herein can provide improved (increased cutting force) at least due to the shape memory elements 17 imparting a radial force. Particularly when they are used in conjunction with a radial force imparted on the shape memory elements 17 by the expansion member 16, as detailed herein. That is, a combined radial force imparted by the combination of the radial force from the shape memory elements 17 and the radial force from the expansion member 16 can impart more radial cutting force on the target site 14 than other approaches such as those with an absence of shape memory elements. Additionally, the systems and apparatuses herein can permit the use of smaller dimension delivery devices (e.g., smaller catheters) as the individual components can have respective radial cross-sections that are smaller than corresponding radial cross-sections of all-in-one devices (e.g., a cutting balloon including a balloon and at least one scoring member permanently affixed thereto), can permit inclusion of a larger quantity of scoring members while still providing a suitable cross-section of the sheathed scoring element, and/or can permit the use of scoring members that can provide a higher scoring or radial holding force at a target site in a vessel. For instance, the systems and apparatus herein when deployed in vivo can generate a radial force on a target site that is in a range from about 1 atmosphere (atm) to about 14 atm of pressure over a target area, as compared to some traditional devices such as those referenced herein that may be limited to less than or about 1 atm of the same target area. For example, in some embodiments the devices and apparatus herein can generate greater than 1 atm of pressure, greater than 2 atm of pressure, greater than 3 atm of pressure, or greater than 4 atm of pressure (e.g., about 2 atm to about 14 atm, about 5 to about 14 atm, etc.) at a target site to permit thrombus/plaque scoring, whereas the traditional devices that are limited to generating 1 atm or less of pressure do not permit sufficient thrombus/plaque scoring.

Moreover, the systems and apparatuses herein can be utilized in a dynamic manner in vivo, in contrast to some other approaches such as those the use an expandable balloon (e.g., with blades affixed to an outer surface thereof) to disrupt (e.g., crack) a lesion (e.g., a calcified lesion). For instance, once deployed at or proximate to a target site in vivo, the systems and apparatuses herein can be moved proximally (e.g., while a deployment sheath is in a scoring condition and the shape memory elements are in an expanded condition), thereby providing an additional force vector (e.g., substantially in a proximal direction) in addition to the radial force imparted by the scoring members on the target site. This dynamic method of deployment of the systems and apparatuses (catheters) herein can yield more uniform and/or effective scoring or cutting of a lesion. For instance, the dynamic method of deployment can yield scoring or cracking of a lesion along a greater portion or an entire longitudinal length of the lesion, as compared to other approaches such as those that employ substantially static or fixed cutting balloons.

The sheathed scoring element 11 can include a deployment sheath 15 (i.e., sheath), a plurality of shape memory elements 17, and a plurality of scoring members 37, as described herein. The deployment sheath 15 can be an outer or axial most member relative at least to the shape memory elements 17 and the scoring members 37. In some embodiments, the deployment sheath 15 can be manifested as a tubular member. The tubular member can be a circular, oval, or other shaped element having a substantially uniform cross-section (e.g., a substantially uniform radial and/or longitudinal cross-section). The deployment sheath 15 can be formed of a polymer or metal, in some instances. For example, the deployment sheath 15 can be formed of a metal such as stainless steel and/or another type of metal. However, in some embodiments, the deployment sheath 15 can be a flexible polymeric sheath, such as an elastomeric polymer sheath.

The deployment sheath 15 can have a smooth surface (e.g., a smooth outer surface that is a radial outermost surface of the deployment sheath 15 and/or an inner surface defining a lumen of the deployment sheath 15 and/or can have substantially uniform dimensions along a length of the deployment sheath 15). Employing a deployment sheath 15 with a smooth surface can promote the deployment sheath 15 to readily move. For instance, the deployment sheath 15 can be configured to longitudinally translate (i.e., axially slide) relative at least to the shape memory elements 17 and a catheter shaft 18, disposed therein. For example, the deployment sheath 15 can selectively be moved (e.g., by actuation of a handle such as those described herein) to selectively constrain (e.g., impart a radial compression force on) the shape memory elements 17 in a constrained condition within the deployment sheath 15, as described herein, or can be moved axially to permit the shape memory elements 17 be exposed from the distal end of the deployment sheath 15 to self-expand axially to an expanded condition, as described herein. For example, the deployment sheath 15 can be configured to move relative to each scoring member in the sheathed scoring element 11. In some embodiments, the deployment sheath 15 can be configured to move relative to the scoring element 11 between a first (e.g., delivery) condition where at least a portion of the shape memory elements 17 or an entirety of the shape memory elements 17 is located in the deployment sheath 15 (e.g., such that the sheath 15 covers, surrounds, or overlays at least the portion of the shape memory elements 17) and a second (e.g., scoring) condition where at least a portion of the shape memory elements 17 or all of the shape memory elements 17 is located distal to a distal end of the deployment sheath 15, and thus at least the portion of the shape memory elements 17 is exposed from the deployment sheath 15 and is configured to permit contact with a target site (e.g., a scoring element on the shape memory element contacts the target site) when the shape memory elements 17 are in an expanded condition), as detailed herein.

The deployment sheath 15 can initially be configured in the first (e.g., delivery) condition and can subsequently be moved (e.g., relative to the scoring member) to the second (e.g., scoring) condition in vivo. For instance, the deployment sheath 15 can be moved from the delivery condition to the scoring condition in vivo when the sheathed scoring element 11 is positioned at or proximate to the target site 14. In some embodiments, the deployment sheath 15 can be retracted proximally relative to the shape member elements 17 (and thus the sheathed scoring element 11) from the delivery condition to the scoring condition by actuation of a handle or other mechanism coupled to the sheathed scoring element 11 (e.g., coupled to a proximal end or proximal end region of the sheathed scoring element 11). In some embodiments, the deployment sheath 15 can be manifested as an elongated tube, as illustrated in FIGS. 1A-1B. The deployment sheath 15 can be coupled to a handle (not illustrated). Actuation of the handle can cause the deployment sheath 15 to move. Examples of suitable deployment sheaths and handles are described in U.S. patent number 11,980,557, the entire content of which is herein incorporated by reference.

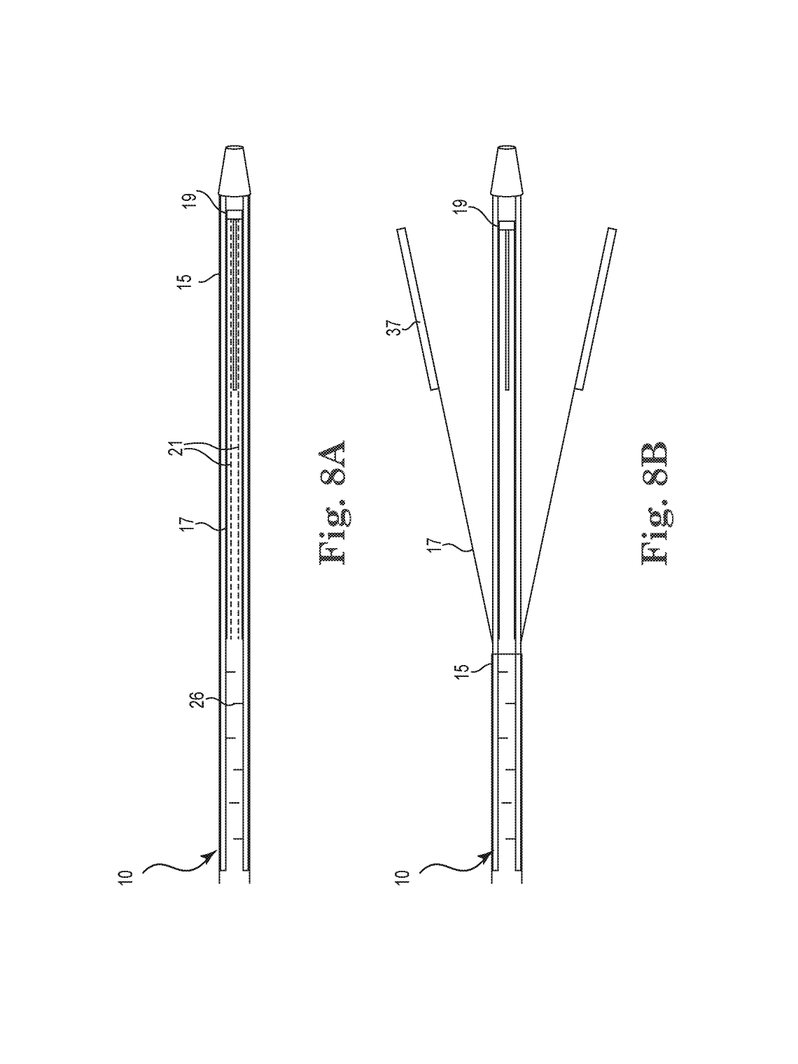

The sheathed scoring element 11 includes the plurality of shape memory elements 17. The shape memory elements 17 can each be the same size and/or the same shape. For instance, the shape memory elements 17 can each be manifested as respective substantially longitudinally extending fingers that are the same size and the same shape, as illustrated in FIGS. 1A-1B. A portion of or all of the shape memory elements 17 can be formed of a shape memory material. For instance, in some embodiments, an entirety of the shape memory elements 17 can be formed of a shape memory material. Examples of suitable shape memory materials include nitinol along with other types of shape memory materials such as those (e.g., MP35-N) described herein. For instance, in some embodiments, an entirety of each of the shape memory elements 17 can be formed of nitinol. In some embodiments, the shape memory elements 17 can be formed of a portion of an elongated tubular member. For example, the shape memory elements 17 can be formed of portions of a hypotube including substantially longitudinally extending slots or openings, as detailed herein with respect to FIGS. 8A-8B. In such embodiments, the hypotube can be formed of a shape memory material or a different material (e.g., other than a shape memory material and which can be radially expanded via an expansion member such as those described herein). For instance, at least the portions of the hypotube corresponding to the shape memory elements 17 can be formed of a shape memory material. In some embodiments, the hypotube can be a circular, annular, or other cross-sectional shape.

In some embodiments, the shape memory elements 17 can be heat-set to a predefined diameter (e.g., when in a radially self-expanded condition) extending radially outward beyond an outer diameter of the deployment sheath 15. The predefined diameter can correspond to or be based on a diameter of a lumen at a target site. As such, the approaches herein can tailor a radial force imparted by the shape memory elements 17 to a particular diameter at a target site. Tailoring the radial force of the shape memory elements 17 to a particular diameter of a target site can promote aspects herein such as imparting a radial force at the target site that can (alone or in combination with a radial force imparted by an expansion member, as detailed herein) score or otherwise remediate the target site, while also mitigating a possibility of perforating a vessel wall at that target site. In some embodiments, the shape memory elements 17 and scoring members 37 disposed thereon can be configured to prevent excessive vessel wall penetration at a target site and otherwise promote control of a depth of cuts from the scoring members 37, as detailed herein.

The shape memory elements 17 (e.g., fingers, legs, struts, etc.) can be configured to automatically actuate upon unsheathing from the sheath 15 (e.g., the outer sheath), thereby moving or expanding to their preset radial diameter greater than the outer diameter of the deployment sheath 15 due to their superelastic properties. For instance, in some embodiments the shape memory elements 17 can be cantilevered shape memory elements. The cantilevered shape memory elements can have a proximal end thereof coupled to the catheter shaft 18 and a distal end thereof that is floating (e.g., is not coupled to the catheter shaft 18). Stated differently, the cantilevered shape memory elements 17 can have only the proximal ends thereof coupled to the catheter shaft. For instance, as illustrated in FIG. 2A, the proximal end of the cantilevered shape memory elements 17 can be coupled to a proximal collar 33 while the distal end of the shape memory elements can float freely. In such embodiments, the proximal end of the shape memory elements 17 can be configured to remain substantially at the same radial position (e.g., relative to longitudinal axis of the catheter 10) while the distal end of the shape memory elements 17 can be configured to automatically expand radially responsive to being unsheathed from the deployment sheath 15, as illustrated in FIG. 1B. Hence, the shape memory elements 17 can be configured to move (e.g., expand radially) independent of or relative to the catheter shaft 18. For instance, the shape memory elements 17, as described herein, can be formed of heat set nitinol that is configured or predisposed to a radially expanded condition. In such instances, the shape memory elements 17 vary in a degree of radial expansion, for instance, as the catheter 10 is moved within different portions, shapes, or sized lumens or cavities in vivo, thereby promoting consistent contact with tissue in the different portions, shapes, and/or sized lumen or cavities in vivo.

The shape memory elements 17 can include a plurality of scoring members 37 disposed thereon. The scoring members 37 can be manifested as blades or as cutting wires. For instance, the shape memory elements 17 can each include an individual scoring member 37 in the form of a substantially longitudinally extending cutting blade, as illustrated in various Figures herein. The scoring members 37 such as cutting blades can be non-movably coupled to the shape memory elements 17. The scoring members 37 can be affixed to an outer surface (e.g., radial most surface) of the shape memory elements 17. The scoring members 37 can be affixed to the shape memory elements 17 via a variety of potential mechanisms including welding and/or adhesive bonding, among other possible techniques. In some embodiments, the scoring members 37 can be coupled to the shape memory elements 17 at the same respective positions. For instance, each of the scoring members 37 can be coupled to an outer surface of the shape memory elements 17 at the same respective position (e.g., the same longitudinal and same circumferential position) about the shape memory elements 17.

In some embodiments, the shape memory elements 17 can comprise pairs of diametrically opposed shape memory elements. In some embodiments, a quantity of the shape memory elements 17 (e.g., “N” shape memory elements) can be equal to or greater than a quantity of the scoring members 37. For instance, in some embodiments a quantity of the shape memory elements 17 (e.g., “N” shape memory elements) can be equal to a quantity of the scoring members (e.g., “N” scoring members). For instance, each shape memory element 17 can include at least one scoring member 37 disposed thereon. For example, each shape memory element 17 can have one, two, or three scoring members 37 disposed thereon while still permitting the application of a sufficient cutting or scoring force via the respective scoring member(s) disposed on each shape memory element 17.

In some embodiments, the scoring members 37 can be offset proximally a distance 39 from a distal tip of the shape memory elements 17, the scoring members 37 are offset distally a distance from a proximal end of the shape memory elements, or both. For instance, in some embodiments, the scoring members 37 can be offset at least proximally a distance 39 (a first distance) from a distal tip of the shape memory elements 17. For instance, as illustrated in FIG. 1B, each of the scoring members 37 can be offset proximally the same distance 39 from the distal tip 41 of the scoring members 37. Similarly, as illustrated in FIG. 1B, each of the scoring members 37 can be offset distally a (second) distance (e.g., the same distance or a different distance) from the proximal end of the scoring members 37. Having the scoring members 37 be offset proximally a distance from at least the distal tip 41 of the shape memory elements 17 can promote aspects herein such as providing a uniform cutting or scoring force and/or permitting the distal tip 41 to provide an additional location for cutting or scoring a target site 14. That is, in some embodiments, the distal tip 41 of the shape memory elements 17 can terminate in a sharp or pointed configuration such that the distal tip is a pointed distal tip that (in addition to the scoring members 37) can score or cut (e.g., incise or dissect) tissue at a target site 14.

In some embodiments, some or all of a plurality of scoring members 37 can have the same shape and/or size. For instance, the scoring members 37 can each be blades (fixed blades coupled to an outer surface of the shape memory elements 17) that are the same size, the same shape, and configured in substantially the same orientation (e.g., are configured in a substantially longitudinal direction).

The shape memory elements 17 and the scoring members 37 can be configured to create uniform substantially longitudinally extending perforations along diseased vessels while maintaining controlled cutting depth through their engineered width. For example, the shape memory elements 17 can each be configured with at least a substantially planar outer surface that the scoring members 37 are coupled to. For instance, each of the shape memory elements 17 can be configured as substantially longitudinally extending rectangular fingers including a substantially planar outer surface. In such embodiments, each of the rectangular fingers can be the same size and shape. Each of the shape memory elements 17 can have at least one respective scoring member 37 (e.g., a scoring blade) disposed on an outer surface (axial most surface) of the shape memory elements 17 (e.g., rectangular fingers). Each of the scoring members 37 can extend a distance axially from the outer surface of the shape memory elements 17. Stated differently, the scoring members 37 (e.g., blades) can protrude a distance from the outer surface of the rectangular fingers. For instance, each of the scoring members 37 can be configured to protrude the same distance from a respective shape memory element 17 that the scoring member 37 is coupled to. Having the scoring members 37 protrude or be offset radially a distance (e.g., the same distance) from the outer surface of the shape memory elements 17 (e.g., fingers) can promote aspects herein, such as imparting a uniform cutting depth and/or control of the cutting depth of the scoring members 37.

For example, the scoring members 37 may cut or score into tissue at least a portion or the entire distance that the scoring members 37 protrude from the outer surface of the shape memory elements. In such instances, the tissue at a target site adjacent that scoring members 37 can contact a portion of the outer surface of the shape memory elements 17 that is adjacent to the scoring members 37. In this way, the shape memory elements 17 can act as a depth guide to permit scoring or cutting at a target site to a depth that is up to by not greater than the distance that the scoring member 37 extends from the outer surface of the shape memory elements 17. Stated differently, the scoring members 37 can penetrate into the tissue a depth that is equal to or is less than the distance that that scoring members 37 extend from the outer surface of the shape memory elements 17, but the shape memory elements 17 themselves may not penetrate or cut into the tissue, in some embodiments. Hence, the systems and apparatuses herein can yield enhanced depth control of cutting or scoring of the target site 14. For instance, the perforations can be created by proximally retracting the apparatus 10 while at least the scoring members 37 are in contact with the target site 14. The creation of such perforations (e.g., microdissections) can enhance vessel compliance and can, in some embodiments, facilitate restenosis (e.g., of an implanted stent) and/or can facilitate more effective drug uptake (e.g., via a subsequently delivered drug-eluting balloon to the target site 14), for instance, by increasing the total surface area of the vessel lumen.

The scoring members 37 may be attached to shape memory elements 17 by various process such as welding, laser bonding, soldering, brazing, adhesive bonding, by using a mechanical fitting or connector, and the like, or in any other suitable way. Moreover, embodiments that include a plurality of scoring members may include a plurality of the same or similar scoring members or a combination of differing scoring member embodiments (e.g., with differing thicknesses, differing shapes, and/or differing orientations, etc.), including any of those described herein. In some embodiments, the scoring members may take a different shape such as a triangular shape where an apex of the triangle is configured to extend radially. In such instances, the triangular shaped scoring blades can reduce an amount of surface area of the scoring member (e.g., the apex of the triangle) in contact with the target site 14 and thereby can provide a further improvement in an amount of scoring force imparted by the scoring wire on the target site 14. In such embodiments, a base side of the triangle can be positioned toward a longitudinal axis of the systems and apparatuses herein. Of course, other shapes can be used in various forms of scoring members without departing from the spirit of the disclosure. For example, various embodiments of scoring members may have circular, square, rectangular, polygonal, or any other suitable cross-sectional shape. The degree of curvature, pattern of curvature, and positioning of curves along the length of scoring members may also vary to include essentially any appropriate configuration.

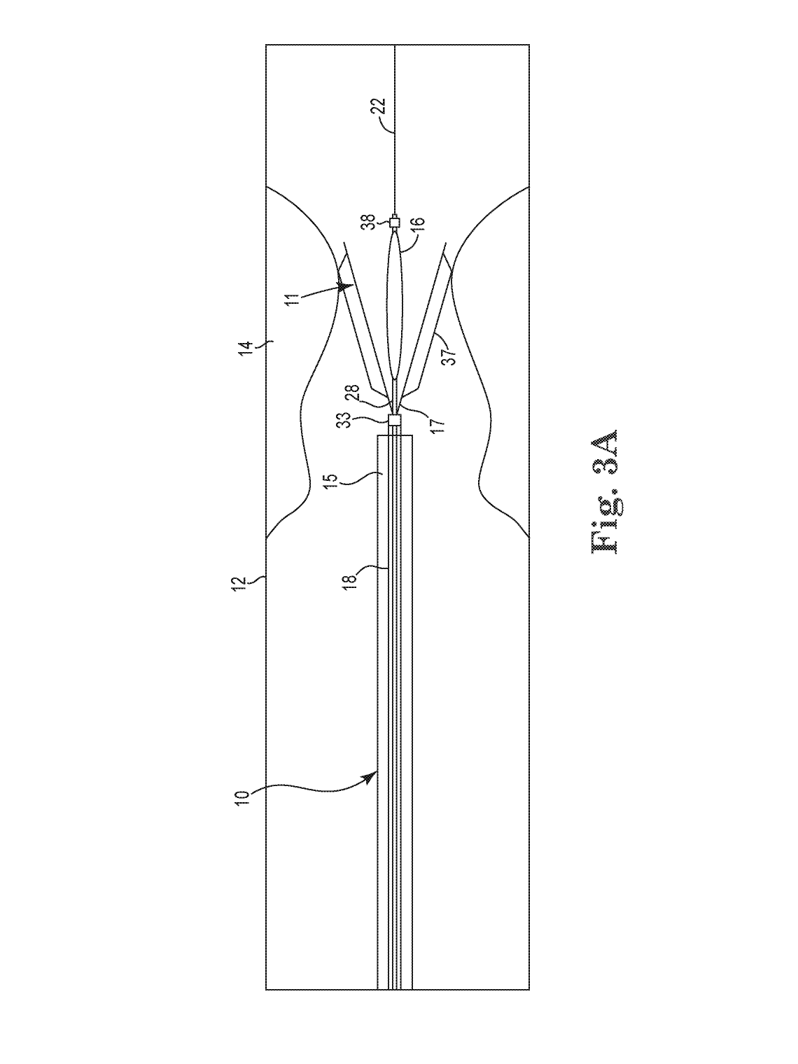

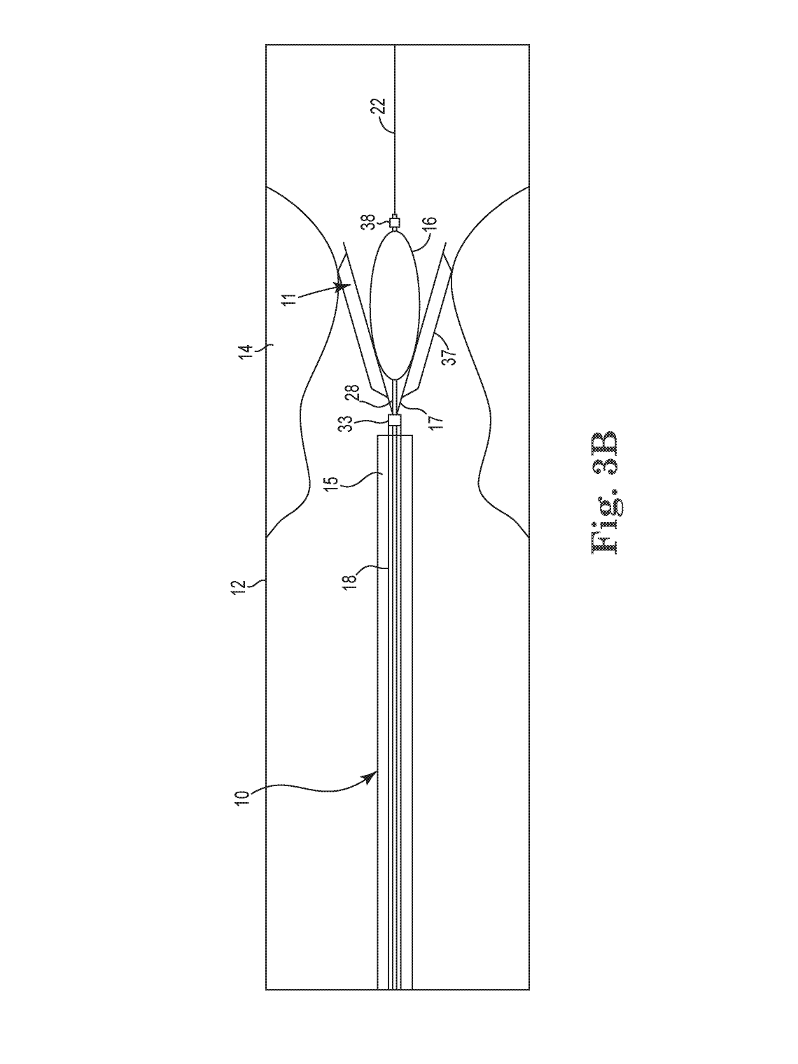

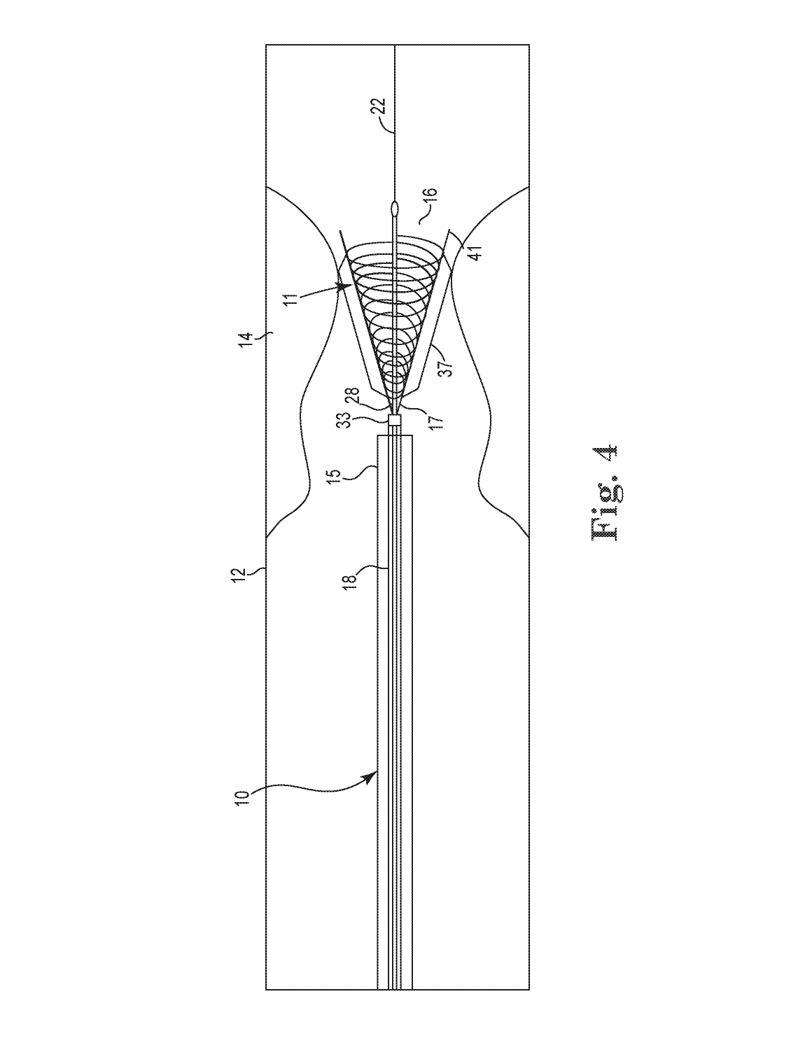

In some embodiments, the catheter 10 can include the sheathed scoring element 11 and can additionally include an expansion member 16. The expansion member 16 can be configured to radially expand in vivo. For example, the expansion member 16 can be a self-expanding expansion member (e.g., a basket or coil formed of a shape memory material) or can be actuated or inflated to expand (e.g., an inflatable balloon or an actuatable basket or coil formed of a shape memory material or a material other than a shape memory material), as described herein. In some embodiments, the expansion member 16 can be manifested as an individual component. In some embodiments, the expansion member 16 can be manifested as a basket, a coil (e.g., a coil formed of a single wire or filament), a scaffold, or can be a balloon. For instance, the expansion member 16 can be a basket (e.g., formed of a shape memory material) that is configured to self-expand or automatically expand e.g., as illustrated in FIGS. 2A-2B, can be a balloon (e.g., a balloon coupled to a catheter shaft defining an inflation lumen) e.g., as illustrated in FIGS. 3A-3B, or can be a coil e.g., as illustrated in FIG. 4, as described herein. However, other shapes, sizes, quantities, and/or types of expansion members are possible. For instance, in some embodiments, the expansion member can be a scaffold such as a scaffold formed of a shape-memory material. The scaffold can have a tubular mesh structure with a uniform or varying (e.g., peak and valley, tapered, etc.) cross-section (e.g., taken at any point along a longitudinal axis) of the scaffold.

FIG. 2A is a simplified partial longitudinal view of the example catheter of FIG. 1B with an example expansion member (basket) in a constrained condition and FIG. 2B is a simplified partial longitudinal view of the example catheter of FIG. 1B with the example expansion member (basket) in an expanded condition. Having the expansion member be radially expandable from a constrained (unexpanded delivery) configuration to an expanded condition in vivo can promote aspects herein such as providing an increased radial force when in the expanded condition to promote cutting or scoring via the scoring elements, and yet can desirably yield a relatively small radial profile of the catheter (e.g., as compared to a solid or fixed geometry expansion component or tip member) in a delivery condition. As illustrated in FIG. 2A, the expansion member 16 can be manifested as a basket. The basket can be formed of one or more wires or filaments. For example, the basket can be a knitted, mesh, or braided basket. For instance, the basket can be a mesh basket that is formed of a plurality of wires or filaments. The basket, like the other expansion members as detailed herein, can be configured to exert a radial force on an innermost surface of the shape memory elements 17. The basket can be a self-expanding basket or can be expandable via actuation of the basket. For instance, the basket can have a first end (e.g., a distal end) fixed to a portion of an inner shaft 28 and can have a second opposing end (e.g., a proximal end) that overlays or floats relative to another portion (e.g., a more proximal portion) of the inner shaft 28. Hence, the portion of the basket that floats over the inner shaft 28 can move about (e.g., longitudinally translate along) the inner shaft 28. In such instances, the inner shaft 28 and the basket may be delivered via a lumen of the catheter shaft 18 to a target site with the basket in an unexpanded or collapsed delivery condition. The basket can then be deployed from the collapsed delivery condition to an expanded condition by pushing or pulling the floating end of the basket longitudinally toward the other fixed opposing end of the basket. For instance, the inner shaft 28 can be translated distally or proximally (e.g., relative to the catheter shaft 18) to push or pull the floating end of the basket longitudinally toward the other fixed opposing end of the basket. In such instances, at least a portion of the basket may be located distal to the scoring members when the basket is in the initial unexpanded delivery configuration, as illustrated in FIG. 2A. In such instances, a majority of or the entirety of the expansion member 16 (e.g., the basket, the coil, or the balloon) may be located distal to the distal end of the shape memory elements 17 and scoring members 37 when the expansion member 16 and the sheathed scoring element 11 (including the shape memory elements 17 and scoring members 37) are in the initial unexpanded delivery configuration within the deployment sheath 15. The basket can have a first (longitudinal) length and a first width (at a widest portion of the basket along the longitudinal axis of the basket) when in the unexpanded condition. Responsive to actuation of the basket (e.g., causing a proximal end of the basket to translate toward a fixed distal end of the basket or causing a distal end of the basket to translate toward a fixed proximal end of the basket) the portion of the basket located distal to the shape memory elements 17 and scoring members 37 may move proximally to be disposed within and in physical contact with the shape memory elements 17 and/or scoring members 37 (e.g., to exert a radial force on the scoring members), as illustrated in FIG. 2B. The basket can have a second length and a second width when in the expanded condition. The second length can be less than the first length and the second width can be greater than the first width.

However, in some embodiments the basket can be configured as a self-expanding basket. For instance, the proximal and distal ends of the basket can be coupled to respective portions of the catheter shaft, while a portion of the basket extending between the proximal and distal ends of the basket can float relative to the catheter shaft thereby permitting the portion (e.g., intermediate portion) of the basket to radially self-expand (e.g., when the sheath is no longer constraining the basket). However, as mentioned in some embodiments one end of the basket can be coupled to a portion of a shaft such as the inner shaft 28 and the opposing end of the basket can float relative to the shaft (e.g., relative to the inner shaft 28). The basket can be formed partially or entirely of a shape memory material, such as those described herein. For example, the basket can be formed entirely of a shape memory material. Employing a basket formed of a shape memory material (e.g., entirely formed of a shape memory material) can promote aspects herein, such as easing delivery and/or uniformly applying an additional amount of radial force via the scoring members to a target site, as described herein. In some embodiments, the basket can be formed of a shape memory material such as nitinol that is heat set to a radially expanded condition. For instance, in FIG. 2A the basket is configured in a constrained condition, while in FIG. 2B, the basket is in an expanded condition. The basket can be formed of a shape memory material such that the basket is configured to self-expand or automatically expand to the expanded condition. Alternatively, or in additional, actuation of a portion of the basket (e.g., moving a distal tip of the basket proximally toward a fixed or stationary a distal end of the basket) can cause the basket to radially expand along at least a portion of the longitudinal length of the basket, as described herein.

As mentioned, the basket can be expanded with or without the presence of an actuation member. For instance, in some embodiments at least a portion of the radial expansion of the basket can be attributable to a force imparted by an actuation member on the basket. While FIGS. 2A-2B illustrate the expansion member 16 as a basket, the disclosure is not so limited. For instance, FIG. 3A is a simplified partial longitudinal view of the catheter of FIG. 1B with another example expansion member 16 in the form of a balloon in a constrained (e.g., a radially constrained) condition and disposed in the blood vessel, while FIG. 3B illustrates a simplified partial longitudinal view of the example catheter 10 of FIG. 3A with the expansion member 16 in the form of the balloon in an expanded condition (e.g., a radially expanded condition).

That is, in some embodiments the catheter 10 (e.g., an apparatus) may include an expansion member 16 in the form of a balloon. The balloon can be coupled to or configured to extend through a lumen of the catheter shaft 18. For instance, the catheter shaft 18 can include an inner shaft 28 extending through a lumen of the catheter shaft 18 (e.g., an outer shaft). In some instances, a distal end of the inner shaft 28 can be located distal to a distal end of the catheter shaft 18 (e.g., outer shaft). In such instances, a guidewire lumen and guidewire, as detailed herein, can extend through a lumen of the inner shaft 28. The inner shaft 28 can be configured to translate relative to the catheter shaft 18. For instance, the inner shaft 28 and the catheter shaft can be configured with complimentary annular profiles to permit the inner shaft 28 to move (e.g., translate) along catheter shaft 18. As detailed herein, the expansion member 16 (e.g., balloon) can be mounted to the inner shaft 18. For instance, at least a distal end or a proximal end of the expansion member 16 (e.g., balloon) can be secured to the inner shaft 28 with the other end secured to a distal collar 38. The distal collar 38 hence can function to attach the distal end of the expansion member 16 to the inner shaft 28. Similarly, in some embodiments a proximal collar or other type of attachment mechanism may be present to attach a proximal end of the expansion member to the inner shaft 28. In other instances, the proximal and/or distal end of the expansion member 16 (e.g., balloon) may be directly secured to the inner shaft 18 without a collar or secured to the inner shaft via another structure. When the expansion member 16 is manifested as a balloon, the balloon can be a compliant, non-compliant, or semi-compliant balloon. For instance, in some embodiments the balloon can be a semi-compliant balloon or non-compliant balloon. In some embodiments that balloon can be a non-compliant balloon. Employing a non-compliant balloon can promote aspects herein such as promoting the radial expansion of at least a portion (e.g., a body portion) of a scoring member and thereby causing the scoring member to contact or score a target site 14. In various embodiments, the balloon can be a substantially cylindrical or spherical balloon, among other possible shapes.

The balloon can be made from typical angioplasty balloon materials including polymers such as polyethylene terephthalate (PET), polyetherimide (PEI), polyethylene (PE), etc. Some other examples of suitable polymers, including lubricious polymers, may include polytetrafluoroethylene (PTFE), ethylene tetrafluoroethylene (ETFE), fluorinated ethylene propylene (FEP), polyoxymethylene (POM), polybutylene terephthalate (PBT), polyether block ester, polyurethane, polypropylene (PP), polyvinylchloride (PVC), polyether-ester (for example, a polyether-ester elastomer such as ARNITEL® available from DSM Engineering Plastics), polyester (for example, a polyester elastomer such as HYTREL® available from DuPont), polyamide (for example, DURETHAN® available from Bayer or CRISTAMID® available from Elf Atochem), elastomeric polyamides, block polyamide/ethers, polyether block amide (PEBA, for example, available under the trade name PEBAX®), silicones, MARLEX® high-density polyethylene, MARLEX® low-density polyethylene, linear low density polyethylene (for example, REXELL®), polyetheretherketone (PEEK), polyimide (PI), polyphenylene sulfide (PPS), polyphenylene oxide (PPO), polysulfone, nylon, perfluoro(propyl vinyl ether) (PFA), other suitable materials, or mixtures, combinations, copolymers thereof, polymer/metal composites, and the like. In some embodiments, it may be desirable to use high modulus or generally stiffer materials so as to reduce balloon elongation. The above list of materials includes some examples of higher modulus materials. Some other examples of stiffer materials include polymers blended with liquid crystal polymer (LCP) as well as the materials listed above. For example, the mixture can contain up to about 5% LCP. Alternatively, the balloon may be coated with a relatively lubricious material such as a hydrogel or silicone.

In some embodiments, the expansion member 16 can be manifested as a coil e.g., a wire coil. For instance, FIG. 4 is a simplified partial longitudinal view of a system for treatment of the vessel lesion including the example catheter, with the example sheathed scoring element including the shape memory elements in the scoring condition, and an example expansion member (a coil) in an expanded condition. The expansion of the coil can be triggered by applying axial force such as either by pushing or pulling on one end of the coil or by self-expanding properties using superelastic materials like nitinol. That is, the coil comprising a plurality of turns can be formed of a shape memory material and thus can radially self-expand in vivo or the coil can be formed of another non-shape memory material and can be configured to radially expand upon actuation of the coil. For instance, as illustrated in FIG. 4, the coil can be formed of a shape memory material that self-expands to a radial profile between opposing shape memory elements 17 thereby imparting an additional axial force on the scoring members 37. Alternatively, or in additional, actuation of a portion of the coil (e.g., moving a distal tip of the coil proximally toward a fixed or stationary a proximal end of the coil) can cause the coil to radially expand along at least a portion of the longitudinal length of the coil. The coil can be a helical coil or cylindrical coil, among other possible types of coils. The coil can be coupled to a portion of a catheter shaft such as the inner shaft 28. For instance, a distal end and/or a proximal end of the coil can be coupled to the inner shaft 28. For example, the proximal end of the coil can float on the inner shaft 28 and the distal end of the coil can be coupled to the inner shaft 28, as illustrated in FIG. 4. Similar to the basket, as detailed herein, the coil can be actuated from an unexpanded condition to an expanded condition via translation of the inner shaft 28 (e.g., relative to the catheter shaft 18). Similar to the basket described with respect to FIGS. 2A-2B, a portion (e.g., distal end or distal end region) of the coil can be positioned distal to a distal tip 41 of the shape memory elements 17 when the coil is in an unexpanded condition. The portion of the coil can be positioned within and in contact with the innermost surface of the portion of the shape memory elements 17 responsive to the longitudinal translation of the inner shaft relative to the catheter shaft, as illustrated in FIG. 4 where the coil is in the expanded condition. As illustrated in FIG. 4, a guidewire lumen and a guidewire 22 (disposed within the guidewire lumen) can extend in a substantially longitudinal direction through the coil.

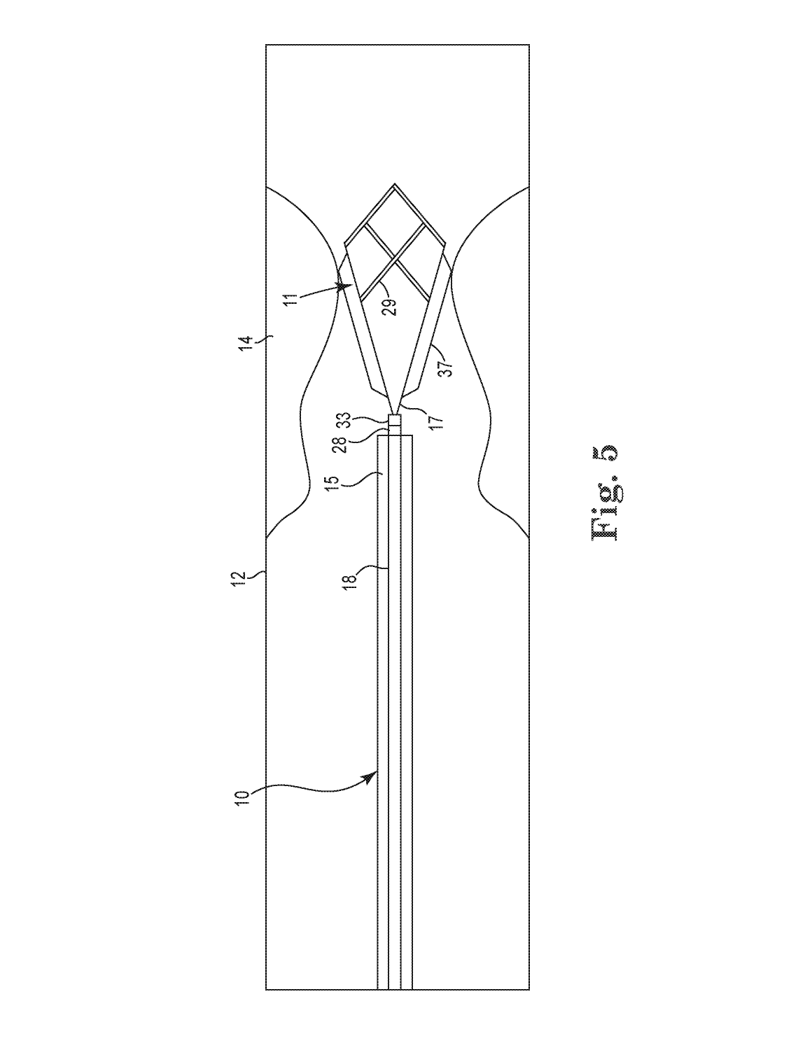

FIG. 5 is a simplified partial longitudinal view of an example catheter 10 with an example sheathed scoring element 11 including reinforced shape memory elements 17 in an expanded condition. As illustrated in FIG. 5, the shape memory elements 17 can be reinforced with one or more struts 29. The struts 29 can provide an additional degree of structural integrity (e.g., reinforcing the shape memory elements 17). The struts 29 can be formed of a shape memory material. For instance, the struts 29 can be formed of a same type of shape memory material (e.g., nitinol) as the shape memory elements 17. The struts 29 can be configured to extend between opposing inner surfaces of opposing shape memory elements 17, as illustrated in FIG. 5. As such, the struts 29 can provide additional axial force and/or may otherwise reinforce the shape memory elements 17 thereby promoting aspects herein e.g., cutting or scoring a lesion.

In some embodiments, some or all of the expansion member 16, the sheathed scoring element 11, and/or the catheter 10 may be manufactured from or may be coated with a lubricious material. Lubricity may be desirable for a number of reasons, such as to enhance the ability of the expansion member 16 and/or the sheathed scoring element 11, to be navigated through the vasculature, particularly when advancing catheter 10 through a relatively narrow or occluded vessel and to minimize friction against ancillary devices such as guide catheters.

In some embodiments, the expansion member 16 and the sheathed scoring element 11 herein do not require or include hinge points such as a hinge point that may typically be employed on commercially available cutting (scoring) blades or atherotomes and which can partially detach the arthrotome/blade from the balloon particularly in tortuous vasculature.

In general, catheter 10 may be advanced over a guidewire 22 through the vasculature to a target site 14. The sheathed scoring element 11 can then be advanced over the guidewire 22 through the vasculature to the target site 14. Prior to and during advancement of the sheathed scoring element 11, the deployment sheath 15 can cover or overlay at least a portion of a scoring member of the sheathed scoring element 11. Once at the target site, the deployment sheath 15 of the sheathed scoring element 11 can be moved (e.g., retracted proximally) to expose a portion of a scoring member of the sheathed scoring element 11 that was covered prior to and during advancement of the sheathed scoring element 11 to the target site. Subsequent to delivery of the sheathed scoring element 11 to the target site 14, the expansion member 16 can be delivered (e.g., advanced over the guidewire 22 and/or through a lumen of the catheter 10) to the target site. For instance, a balloon can be advanced (e.g., over the guidewire 22) through the vasculature to the target site 14. Once at the target site, the balloon can then be inflated (e.g., to an inflated or expanded condition) such that the exterior surface of the balloon contacts and radially imparts a force on the shape memory elements to cause the scoring member to cut/score and expand a target site 14 such as a lesion. The target site may be within any suitable peripheral or cardiac location, for example.

The shaft 18 may be a catheter shaft, similar to typical catheter shafts which have a distal end portion and a proximal end portion. For example, shaft 18 may include an inner tubular member (e.g., defining a guidewire lumen to accommodate a guide wire and track over it to the target lesion) and outer tubular member (e.g., defining an inflation lumen to allow passage of inflation media, usually contrast dye diluted with saline solution). Tubular members may be manufactured from a number of different materials. For example, tubular members may be made of metals, metal alloys, polymers, metal-polymer composites or any other suitable materials. Some examples of suitable metals and metal alloys include stainless steel, such as 304V, 304L and 316L stainless steel; nickel-titanium alloy such as linear-elastic or super-elastic Nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), Hastelloy, Monel 400, Inconel 825, or the like; or other suitable material. Some examples of suitable polymers include those described above in relation to the balloons. Of course, any other suitable polymer may be used without departing from the spirit of the disclosure. The materials used to manufacture inner tubular member may be the same as or be different from the materials used to manufacture outer tubular member.

Tubular members may be arranged in any appropriate way. For example, in some embodiments inner tubular member can be disposed coaxially within outer tubular member. According to these embodiments, inner and outer tubular members may or may not be secured to one another along the general longitudinal axis of shaft 18. Alternatively, inner tubular member may follow the inner wall or otherwise be disposed adjacent the inner wall of outer tubular member. Again, inner and outer tubular members may or may not be secured to one another. For example, inner and outer tubular members may be bonded, welded (including tack welding or any other welding technique), or otherwise secured at a bond point. In some embodiments, the bond point may be generally disposed near the distal end portion of shaft 18 or near a proximal end portion of the shaft 18. However, one or more bond points may be disposed at any position along shaft 18. The bond may desirably impact, for example, the stability and the ability of tubular members to maintain their position relative to one another. In still other embodiments, inner and outer tubular member may be adjacent to and substantially parallel to one another so that they are non-overlapping. In these embodiments, shaft 18 may include an outer sheath that is disposed over one or more tubular members.

Inner tubular member includes an inner lumen. In a preferred embodiment, inner lumen is a guidewire lumen. Accordingly, catheter 10 can be advanced over guidewire 22 to the desired location. The guidewire lumen may extend along essentially the entire length of catheter shaft so that catheter 10 resembles traditional “over-the-wire” catheters. Alternatively, the guidewire lumen may extend along only a portion of shaft 18 so that catheter 10 resembles “single-operator-exchange” or “rapid-exchange” catheters. Regardless of which type of catheter is contemplated, catheter 10 may be configured so that expansion member 16 is disposed over at least a region of inner lumen or is configured to be advanced therethrough. For instance, an expansion member 16 in the form of a balloon may be advanced through the guidewire lumen, through a different lumen (other than the guidewire lumen) in the catheter 10, or may be advanced separately from the catheter 10 (e.g., via a separate balloon catheter). In addition to some of the structures described above, the catheter shaft 18 may also include a number of other structural elements, including those typically associated with catheter shafts.

In some embodiments such as those employing a balloon as an expansion member, an inflation lumen can be in fluid communication with the balloon. For instance, shaft 18 may also include an inflation lumen in fluid communication with the interior of the balloon, that may be used, for example, to transport inflation media to and from the balloon. For example, when an outer tubular member is disposed over an inner tubular member, the inflation lumen may be defined within the space between the outer and inner tubular members. Alternatively, the inflation lumen can be provided in a separate catheter shaft, for instance when the balloon is delivered via a catheter that is separate from the catheter 10. In some embodiments, the balloon can be manifested as a single balloon. In some embodiments, the inflation lumen can be manifested as a single inflation lumen. In some embodiments, the balloon can be a single balloon and the inflation lumen can be manifested as a single inflation lumen in fluid communication with an interior of the balloon. Thus, inflation media delivered through the inflation lumen may be introduced into the interior of the balloon to inflate the balloon (e.g., cause the balloon to shift from a deflated condition to an inflated or expanded condition). The balloon can be inflated to a pressure (e.g., about 1600 kilopascals, etc.) in an inflated condition that is typically associated with inflatable balloons, for example.

In some embodiments, the shape memory elements 17 can be substantially planar shape memory elements, for instance as illustrated in FIGS. 1A-1B. However, the shape memory elements 17 have a different shape in some embodiments.

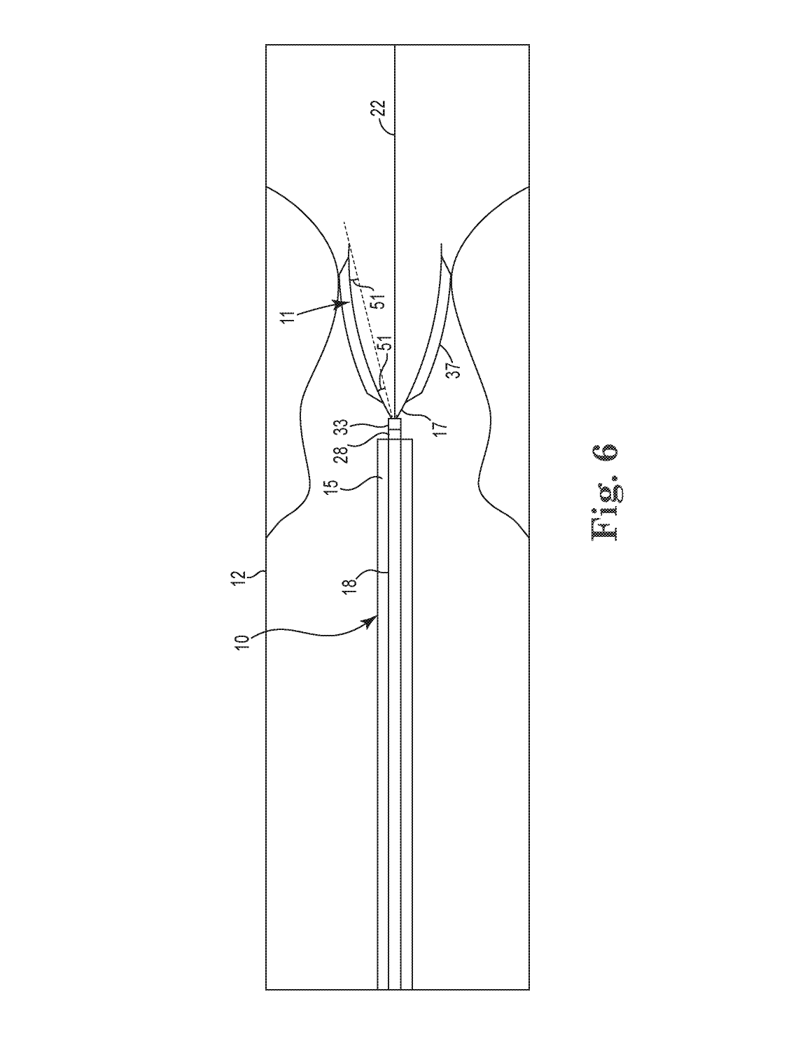

FIG. 6 is a simplified partial longitudinal view of an example catheter 10 including curved shape memory elements 17 in an expanded condition. For instance, as illustrated in FIG. 6, a middle region (located between a distal tip or distal end region and a proximal end region or proximal tip of the shape memory elements 17) can be configured to extend radially outward in a curved manner at a non-zero angle (e.g., as represented by angle 51). The non-zero angle can be any angle in a range from about 5 degrees to about 60. All individual values and sub-ranges from about 5 degrees to about 60 degrees are included. Employing curved shape memory elements can promote aspects herein, such as promoting the scoring members 37 to contact a target site with increased force, etc. For instance, the curved nature of the shape memory elements 17 may increase a propensity of the scoring members 37 to remain in contact with and thereby score or cut tissue or a target site as during a procedure (e.g., as the sheathed scoring element 11, etc. is retraced proximally within in vasculature including the target site 14). While various Figures herein illustrate the scoring members 37 as having a substantially uniform cross-section along at least a portion (e.g., at least a middle region or central portion located between a distal end region and a proximal end region thereof) of the scoring member 37, other shapes and configurations of the scoring members are possible. For instance, the scoring members 37 may be configured with a tapered geometry extending along a longitudinal length (e.g., the entire length) of the scoring members 37. In such instances, the scoring members 37 can be configured with a relatively wide cross-section at a proximal end of the scoring members 37 and can be tapered in a linear or non-linear fashion to a relatively narrow cross-section at the distal end of the scoring members 37, in some embodiments.

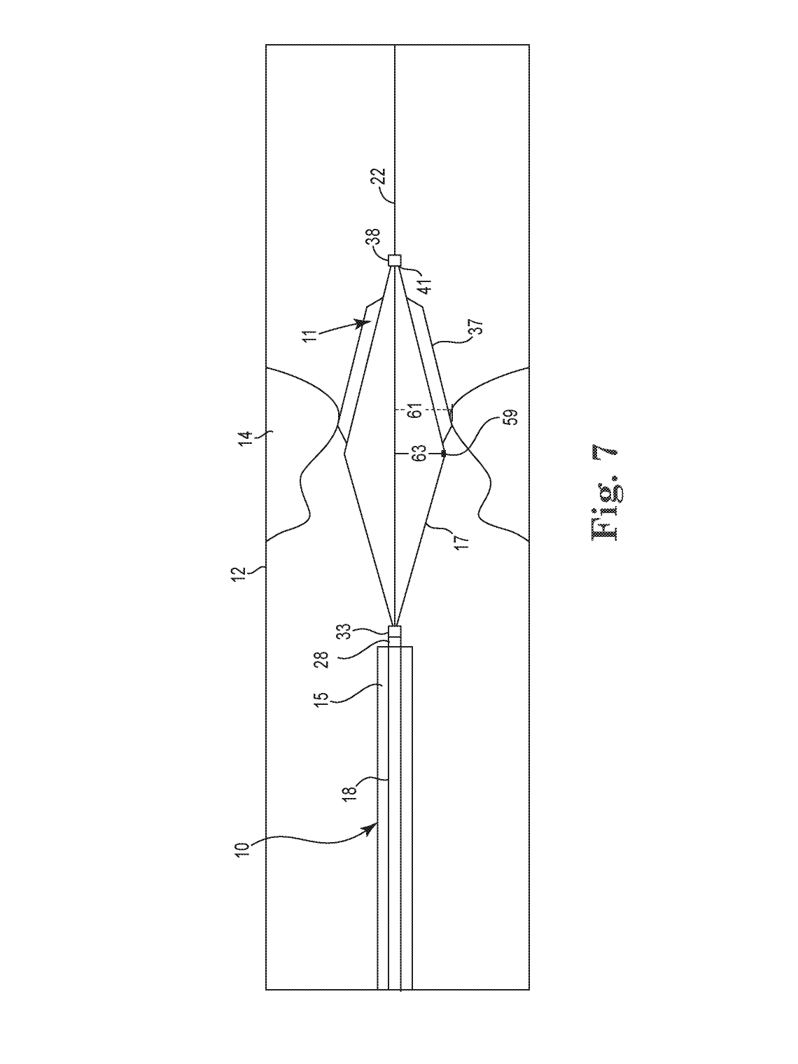

While some embodiments employ shape memory elements that are cantilevered (e.g., with a floating or free proximal end) the disclosure is not so limited. Rather in some embodiments a proximal end and a distal end of the shape memory elements can be coupled to the catheter shaft. For instance, FIG. 7 is a simplified partial longitudinal view of an example catheter including non-linear (e.g., distally and proximally coupled) shape memory elements in an expanded condition. FIG. 7 is analogous to FIG. 1B, with the change that the shape of the shape memory elements 17 is different and that the distal ends of the shape memory elements 17 are coupled to the catheter shaft 18. For instance, as illustrated in FIG. 7, the proximal ends of the shape memory elements can be coupled to the catheter shaft 18 (e.g., are coupled at a proximal collar 33) and the distal ends of the shape memory elements 17 can be coupled to the catheter shaft 18 (e.g., are coupled at a distal collar 38). Similar to FIG. 6, the shape memory elements can be non-linear shape memory elements. However, unlike the curved shape memory elements in FIG. 6, the shape memory elements 17 in FIG. 7 can include an inflection point 59 located (e.g., at a midpoint) between the proximal end and the distal end of the shape memory elements, as illustrated in FIG. 7. In such embodiments, the shape memory elements 17 can include scoring members located thereon at a location that is distal to the inflection point 59 and is proximal to the distal ends of the shape memory elements. Alternatively, or in addition, the shape memory elements 17 can include scoring members (not illustrated) located thereon at a location that is proximal to the inflection point 59 and distal to a proximal end of the shape memory elements. In some embodiments, the scoring members 37 can extend a distance 61 (radially relative to a longitudinal axis of the catheter 10) that is greater than a radial distance 63 (relative to the longitudinal axis of the catheter 10) associated with the inflection point 59, as illustrated in FIG. 7 e.g., to ensure contact of a target site with the scoring members 37.

Various configurations of the shape memory elements 17 are described herein. In any case, the automatic actuation of the shape memory elements 17 promotes predictable and controlled deployment without requiring manual force application. In some embodiments, the shape memory elements 17 themselves can provide at least a portion of an axial force profile imparted by the scoring members 37 at a target site. Additionally, the configuration of the shape memory members 17 can permit an additional axial force to be provided by an expansion member. That is, the shape memory elements 17 can work in concert with an expansion member (e.g., a basket, balloon, coil, or a scaffold) to provide additional force modulation when needed at a target site (e.g., when a calcified lesion is present at the target site). This dual-force capability permits readily and precisely treating varying lesion types, from soft plaque to calcified deposits.

FIG. 8A is a simplified partial longitudinal view of an example catheter 10 including shape memory elements 17 formed as respective portion of an elongated tubular member with the shape memory elements in a constrained condition. As illustrated in FIGS. 8A, in some embodiments the shape memory elements 17 can be manifested as respective cantilevered portions formed monolithically in an elongated tubular member. The cantilevered shape memory elements can be spaced circumferentially about the elongated tubular member. For example, the shape memory elements 17 can be formed of respective portions of a hypotube between substantially longitudinally extending slots or openings 21. The slots or openings 21 can be substantially elongated slots which are spaced apart circumferentially and extend between a first location that is proximal to the distal end of the elongated tubular member substantially axially along a portion of a length of the elongated tubular member to a second location that is proximal to the first location. In such instances, the shape memory elements 17 can be manifested as the solid portions of the elongated tubular member (e.g., a slotted hypotube) that are located between adjacent slots or openings in the elongated tubular member. The solid portions can be cantilevered portions having a proximal end thereof affixed to a proximally adjacent portion of the elongated tubular member, while the distal ends of the cantilevered portions are detached or free from the distal end region 19 of the hypotube. Thus, the cantilevered portions can be configured to move (e.g., deflect radially outward) in a radial manner, for instance, when located in vivo and when no longer constrained to an initial delivery configuration by the sheath 15. For instance, the elongated tubular member (e.g., hypotube) can be formed of a shape memory material or a different material (e.g., other than a shape memory material and which can be radially expanded via an expansion member such as those described herein). For example, at least the portions of the elongated tubular member corresponding to the shape memory elements can be formed of a shape memory material.

As illustrated in FIG. 8A, the shape memory elements 17 can be offset proximally or spaced a distance from the distal tip of the elongated tubular member. Thus, an annular distal end region 19 of the elongated tubular member can have an absence of the shape memory elements 17 and/or an absence of slots formed in the distal end region 19. Thus, the annular distal end region 19 may form a cylindrical portion of the elongated tubular member, defining a portion of the guidewire lumen extending therethrough. The absence of shape memory elements 17 and/or slots in the distal end region 19 can promote aspects herein such as enhancing a structural stability of the catheter 10. As mentioned, a guidewire (not illustrated in FIG. 8A) can extend through a lumen of the catheters herein. For instance, a guidewire can extend through a lumen (e.g., a guidewire lumen) within the elongated tubular member (e.g., a slotted hypotube), including the annular distal end region 19 distal of the radially deflecting shape member elements 17. Hence, one or more components of the catheter 10 illustrated in FIG. 8A can track or move in a substantially longitudinal manner about the guidewire. For instance, the annular distal end region 19 of the elongated tubular member can track along the guidewire.

In some embodiments, the elongated tubular member can be a hypotube such as a hypotube having notches, openings, or slots 26, located proximal to the shape memory elements. Such notches, openings, or slots 26 can provide the elongated tubular member with one or more degrees of freedom of movement, for instance, to ease navigation of the elongated tubular member within the vasculature or body cavity of a patient. As illustrated in FIG. 8A, a sheath 15, such as those described herein, can maintain the shape memory elements 17 in a constrained condition. In such instances, the sheath 15 can be translated proximally to permit the shape memory elements 17 to expand radially to an expanded condition. For instance, FIG. 8B is a simplified partial longitudinal view of the example catheter of FIG. 8A with the shape memory elements 17 are in an expanded condition. Employing shape memory elements formed as respective portions of a hypotube can promote aspects herein such as easing delivery and/or deployment (e.g., radial expansion) of the shape memory elements in vivo.

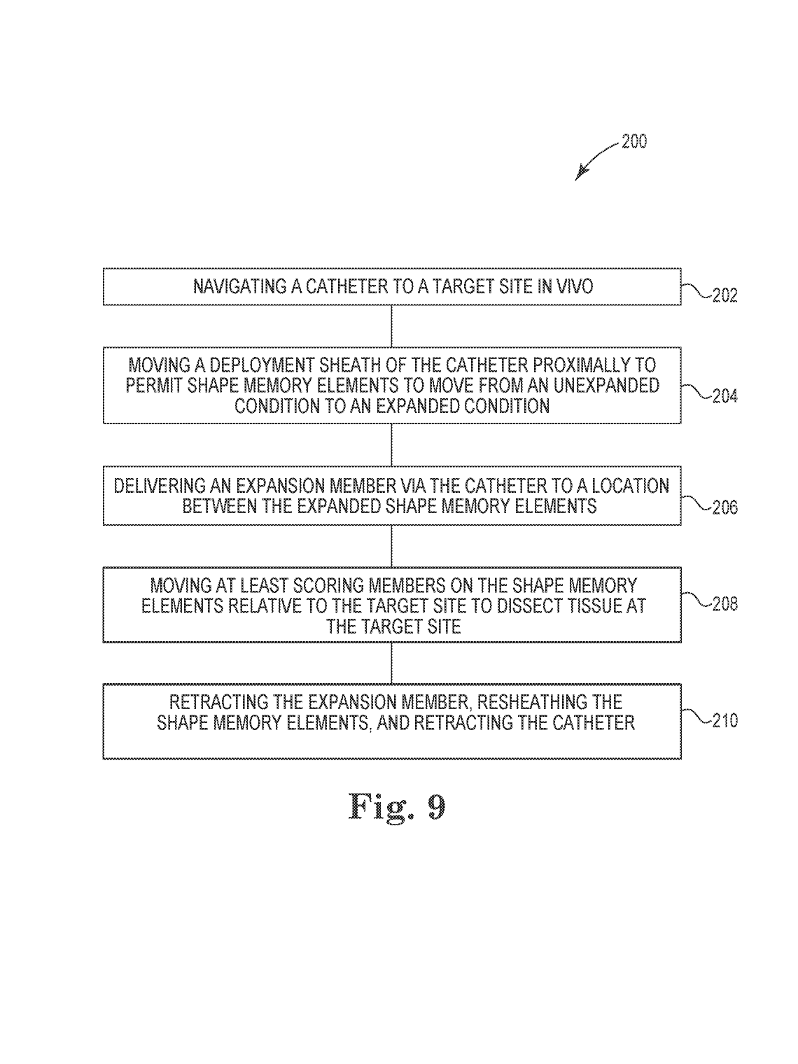

FIG. 9 is an example of a method 200 employing a catheter including sheathed self-expanding shape memory elements with scoring members. The method 200 can be employed with any of the systems and apparatuses previously described with respect to FIGS. 1A-8 herein.

At 202, the method 200 can include navigating a catheter in vivo to a location at or proximate to (e.g., distal to) a target site in a patient. At 204, the method 200 can include moving a deployment sheath (e.g., translating the sheath proximally) relative at least to the scoring members and the shape memory elements. That is, the deployment sheath can be moved from a delivery condition to a scoring condition.

At 206, the method can include delivering an expansion member (e.g., a basket, basket, or coil) via the catheter (e.g., via a lumen of the catheter) to a central location between the shape memory elements. In some embodiments, the expansion member can be manifested as a balloon and can be inflated subsequent to delivery to the central location between the shape memory elements. In some embodiments, the expansion member can be manifested as coil (e.g., formed of a material other than a shape memory material) and can be actuated, as described herein, to undergo expansion subsequent to delivery to the central location between the shape memory elements. In some embodiments, the expansion member can be manifested as a self-expanding basket or a self-expanding coil and can undergo radial expansion automatically (e.g., undergo self-expansion) once delivered to the central location between the shape memory elements. In any case, the expansion member can be configured to expand to impart a radial force on an innermost surface (located most proximate to a longitudinal axis of the catheter) of the shape memory elements thereby increasing a total amount of radial force imparted by the scoring members (in addition to a radial force intrinsically imparted by the shape memory elements) located on the outer surfaces of the shape memory element to a target site.

At 208, the method 200 can include moving at least the scoring members (e.g., and the shape memory elements) relative to the target site. For instance, the method 200 can include proximally translating (retracting) some or all components of the catheter while the scoring members and the shape memory elements in contact with or are located proximate to (e.g., are located distal of) the target site. Hence, the systems and apparatuses herein can be utilized in a dynamic manner in vivo, in contrast to some other approaches such as those the use an expandable balloon (e.g., with scoring blades affixed to an outer surface thereof) to disrupt (e.g., crack) a lesion (e.g., a calcified lesion). For instance, once deployed at or proximate to a target site in vivo, the systems and apparatuses herein can be moved proximally while in an expanded or deployed configuration thereby providing an additional force vector (e.g., substantially in a proximal direction) in addition to the radial force imparted by the scoring members on the target site. This dynamic method of deployment can yield more uniform and/or effective scoring or cutting of a lesion (e.g., along a greater portion or an entire longitudinal length of the lesion) as compared to other approaches such as those that employ substantially static or fixed cutting balloons.

At 210, the method 200 can include removal (retraction) of the expansion member and can subsequently include resheathing of the shape memory elements (e.g., moving the sheath distally to overlay at least a portion of the shape memory elements such as at least the portions of the shape memory elements have the scoring members affixed thereto) to permit readily and safely retracting of the entire catheter from a patient.

In some embodiments, the method 200 can include additional method elements such as injection of a contrast agent or dye. For instance, the contrast agent or dye can be introduced in vivo prior to, during, and/or subsequent to moving at least the scoring members to or proximate to the target site. For example, the contrast dye or agent can be introduced via one or more lumens of the catheters described herein such as introduction via a central lumen of the inner shaft and/or via an annular space between the catheter shaft and the sheath, among other possibilities. In at least some embodiments, portions or all of the medical apparatuses (devices) described herein may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical devices described herein in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical devices described herein to achieve the same result.

In some embodiments, a degree of Magnetic Resonance Imaging (MRI) compatibility is imparted into the medical apparatuses described herein. For example, the medical apparatuses described herein, or portions thereof, may be made of a material that does not substantially distort the image and create substantial artifacts (i.e., gaps in the image). Certain ferromagnetic materials, for example, may not be suitable because they may create artifacts in an MRI image. The medical devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made to various details herein, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A catheter for treatment of a vessel lesion, the catheter comprising:
a catheter shaft; and
a sheathed scoring element including:
shape memory elements that are configured to move between a constrained condition and a radially expanded condition;
scoring members located on a portion of the shape memory elements; and
a deployment sheath that is configured to move relative to the scoring members and the shape memory elements between:
a delivery condition where the scoring members and at least a portion of the shape memory elements are located in the deployment sheath; and
a scoring condition where:
at least the portion of the shape memory elements is located distal to a distal end of the deployment sheath and the shape memory elements are configured in the expanded condition; and
the scoring members are located distal to a distal end of the deployment sheath and are configured to contact a target site.

2. The catheter of claim 1, wherein at least the proximal ends of the shape memory elements are coupled to the catheter shaft.

3. The catheter of claim 2, wherein the shape memory elements further comprise cantilevered shape memory elements with only the proximal ends of the cantilevered shape memory elements coupled to the catheter shaft.

4. The catheter of claim 1, wherein the shape memory elements are formed of shape memory material that is heat set to the expanded condition.

5. The catheter of claim 1, wherein the shape memory elements have a pointed distal tip configured to dissect tissue at the target site.

6. The catheter of claim 1, wherein:
the shape memory elements have the same size and same shape; and
the scoring members have the same size and same shape.

7. The catheter of claim 1, wherein the scoring members are offset proximally a distance from a distal end of the shape memory elements, are offset distally a distance from a proximal end of the shape memory elements, or both.

8. The catheter of claim 1, wherein the scoring members further comprise a scoring wire, a scoring blade, or combinations thereof.

9. The catheter of claim 1, wherein the scoring members are disposed on an outer surface of the shape memory elements, and wherein the outer surface is substantially planar.

10. The catheter of claim 9, wherein the scoring members extend a distance radially from the outer surface of the shape memory elements.

11. The catheter of claim 1, further comprising an expansion member coupled to an inner shaft, wherein the inner shaft is disposed within and is configured to translate longitudinally relative to the catheter shaft, and wherein the expansion member is configured to impart a radial force on an innermost surface of the portion of the shape memory elements when the deployment sheath is in the scoring condition and the shape memory elements are in the expanded condition.

12. The catheter of claim 11, wherein the expansion member is a basket, balloon, a coil, or a scaffold.

13. The catheter of claim 11, wherein the expansion member is configured to expand and impart the radial force on the innermost surface of the portion of the shape memory elements responsive to longitudinal translation of the inner shaft relative to the catheter shaft.

14. The catheter of claim 13, wherein a first end of the expansion member is coupled to the inner shaft and wherein a second opposing end of the expansion member floats on the inner shaft.

15. The catheter of claim 14, wherein a portion of the expansion member is positioned distal to a distal tip of the shape memory elements when the expansion member is in an unexpanded condition, and wherein the portion of the expansion member is positioned proximal to the distal tip and in contact with the innermost surface of the portion of the shape memory elements when the expansion member is in an expanded condition responsive to the longitudinal translation of the inner shaft relative to the catheter shaft.

16. A catheter for treatment of a vessel lesion, the catheter comprising:
a catheter shaft; and
a sheathed scoring element coupled to the catheter shaft, the sheathed scoring element including:
shape memory elements formed of a shape memory material that is configured to move between a constrained condition and a radially self-expanded condition;
scoring members located on a portion of the shape memory elements; and
a deployment sheath that is configured to move relative to the scoring members and the shape memory elements between:
a delivery condition where the scoring members and at least a portion of the shape memory elements are located inside of the deployment sheath; and
a scoring condition where:
at least the portion of the shape memory elements is located distal to a distal end of the deployment sheath and the shape memory elements are configured in the radially self-expanded condition; and
the scoring members are located distal to a distal end of the deployment sheath and are configured to contact a target site; and
an expansion member configured to impart a radial force on the portion of the shape memory elements when the deployment sheath is in the scoring condition and the shape memory elements are in the radially self-expanded condition.

17. The catheter of claim 16, wherein the shape memory elements are cantilevered substantially longitudinally extending portions of an elongated tubular member, wherein the cantilevered substantially longitudinally extending portions are offset proximally a distance from a distal end region of the elongated tubular member.

18. The catheter of claim 16, wherein the scoring members further comprise blades, and wherein the blades are spaced proximally a distance from a distal end of the shape memory elements.

19. A catheter for treatment of a vessel lesion, the catheter comprising:
a catheter shaft; and
a sheathed scoring element coupled to the catheter shaft, the sheathed scoring element including:
shape memory elements that are formed of a shape memory material and are configured to move between a constrained condition and a radially self-expanded condition, wherein the shape memory elements have a substantially planar outer surface;
scoring blades located on a portion of the shape memory elements, wherein the scoring blades are offset at least proximally from distal tips of the shape memory elements and wherein the scoring blades protrude a distance from substantially planar outer surface of the shape memory elements; and
a deployment sheath that is axially movable relative to the scoring blades and the shape memory elements between:
a delivery condition where the scoring blades and at least a portion of the shape memory elements are located inside of the deployment sheath; and
a scoring condition where:
at least the portion of the shape memory elements is located distal to a distal end of the deployment sheath and the shape memory elements are configured in the radially self-expanded condition; and
the scoring blades are located distal to a distal end of the deployment sheath and are configured to contact a target site; and
an expansion member configured to impart a radial force on the portion of the shape memory elements when the deployment sheath is in the scoring condition and the shape memory elements are in the radially self-expanded condition.

20. The catheter of claim 19, wherein each scoring blade of the scoring blades is configured to protrude the same distance from the substantially planar outer surface of the shape memory elements, and wherein each of the shape memory elements is formed of a shape memory material that is heat set to the radially self-expanded condition.
